(19) United States
(12) Patent Application Publication
(10) Pub. No.: US 2026/0157664 A1
(43) Pub. Date: Jun. 11, 2026

(54) BLOOD GLUCOSE MEASURING DEVICE, ELECTRONIC DEVICE, AND OPERATING METHOD THEREOF FOR PROCESSING SIGNALS FOR MEASURING BLOOD GLUCOSE

(71) Applicant: ELECTRONICS AND TELECOMMUNICATIONS RESEARCH INSTITUTE, Daejeon (KR)

(72) Inventors: Jong Deog Kim, Daejeon (KR); Bong Kyu Kim, Daejeon (KR)

(21) Appl. No.: 19/310,049

(22) Filed: Aug. 26, 2025

(30) Foreign Application Priority Data
Dec. 11, 2024 (KR) 10-2024-0183888
Mar. 11, 2025 (KR) 10-2025-0031523

(51) Int. Cl. A61B 5/145; A61B 5/00; A61B 5/1455; G16H 10/40

(52) U.S. Cl. CPC A61B 5/14532; A61B 5/1455; A61B 5/7203; A61B 5/7246; G16H 10/40; A61B 2562/0238

(57) ABSTRACT

The blood glucose measuring device includes a processor and a memory storing instructions, wherein the instructions, when executed by the processor, cause the blood glucose measuring device to obtain, via a first receiver, a plurality of reflection signals, which is a plurality of lights output from the light source, and obtain, via a second receiver, a plurality of transmission signals, determine, for each of the plurality of reflection signals and each of the plurality of transmission signals, a slope efficiency, which represents a ratio of an output change to an input change of the blood glucose measuring device, determine first analysis data based on the slope efficiency of the plurality of reflection signals and/or second analysis data based on the slope efficiency of the plurality of transmission signals, and determine a final blood glucose value based on at least one of the first analysis data or the second analysis data.

### CROSS-REFERENCE TO RELATED APPLICATIONS

This application claims the benefit of Korean Patent Application No. 10-2024-0183888, filed on Dec. 11, 2024, and 10-2025-0031523, filed on Mar. 11, 2025, in the Korean Intellectual Property Office, the entire disclosures of which are incorporated herein by reference for all purposes.

### BACKGROUND

### 1. Field of the Invention

The present disclosure relates to a blood glucose measuring device, an electronic device, and an operating method thereof for processing signals for measuring blood glucose.

### 2. Description of the Related Art

Blood glucose is an important biomarker that indicates the metabolic state of the body and may be used to manage diabetes and monitor health. Various blood glucose measuring devices capable of monitoring biosignals are being developed to continuously measure blood glucose. A blood glucose measuring device may use invasive methods such as finger pricking or non-invasive methods that use various signals such as heat, electromagnetic waves, ultrasound, and the like. Research is actively being conducted to measure blood glucose more accurately using blood glucose measuring devices that use non-invasive methods to measure blood glucose more indirectly than invasive methods.

### SUMMARY

Embodiments provide an electronic device capable of more effectively removing noise from a signal by determining analysis data based on a slope efficiency, which represents an output change according to an input change in a blood glucose measuring device.

Embodiments provide an electronic device capable of more accurately measuring blood glucose in a non-invasive manner by determining a final blood glucose value based on first analysis data and second analysis data, which are based on a slope efficiency of a plurality of reflection signals multiply scattered and reflected in a reverse direction and a plurality of transmission signals multiply scattered and transmitted in a forward direction in a blood glucose measuring device.

According to an aspect, there is provided a blood glucose measuring device including a processor and a memory storing instructions, wherein the instructions, when executed by the processor, cause the blood glucose measuring device to obtain, via a first receiver positioned on a same side as a light source based on a body of a user, a plurality of reflection signals, which is a plurality of lights output from the light source being multiply scattered and reflected in a reverse direction from the body, and obtain, via a second receiver positioned on an opposite side of the light source, a plurality of transmission signals, which is the plurality of lights being multiply scattered and transmitted in a forward direction while passing through the body, determine, for each of the plurality of reflection signals and each of the plurality of transmission signals, a slope efficiency, which represents a ratio of an output change to an input change of the blood glucose measuring device, determine first analysis data based on the slope efficiency of the plurality of reflection signals and/or second analysis data based on the slope efficiency of the plurality of transmission signals, and determine a final blood glucose value based on at least one of the first analysis data or the second analysis data. The plurality of lights has different wavelengths.

The instructions may, when executed by the processor, cause the blood glucose measuring device to determine, for each of the plurality of reflection signals and each of the plurality of transmission signals, a normalized slope efficiency, which represents a relationship between the slope efficiency at a predetermined reference time point and the slope efficiency at a blood glucose measurement time point, and determine the first analysis data and the second analysis data based on the normalized slope efficiency.

The instructions may, when executed by the processor, cause the blood glucose measuring device to determine the first analysis data based on a first correlation between respective normalized slope efficiencies of the plurality of reflection signals and determine the second analysis data based on a second correlation between respective normalized slope efficiencies of the plurality of transmission signals.

The first correlation may be a difference between logarithmic values of the respective normalized slope efficiencies of the plurality of reflection signals.

The second correlation may be a difference between logarithmic values of the respective normalized slope efficiencies of the plurality of transmission signals.

The first correlation may be a sum of logarithmic values of the respective normalized slope efficiencies of the plurality of reflection signals.

The second correlation may be a sum of logarithmic values of the respective normalized slope efficiencies of the plurality of transmission signals.

The instructions may, when executed by the processor, cause the blood glucose measuring device to determine the first correlation and the second correlation based on a degree of blood glucose correlation according to the wavelengths of the plurality of lights.

The instructions may, when executed by the processor, cause the blood glucose measuring device to determine a variance error of a first prediction value and a variance error of a second prediction value, wherein the first prediction value and the second prediction value are derived from the first analysis data and the second analysis data, respectively, and determine one of the first prediction value and the second prediction value of which the variance error is less than the other as the final blood glucose value.

The final blood glucose value may be corrected by an electronic device that learns, using correction data comprising a blood glucose value measured invasively and a measurement time point, a difference between the final blood glucose value and the blood glucose value in the correction data to determine a corrected blood glucose value.

The input change may be a change in a current intensity applied to the light source, and the output change may be a change in a plurality of reflection signals and a plurality of transmission signals corresponding to the current intensity.

According to an aspect, there is provided an electronic device including a processor and a memory storing instructions, wherein the instructions, when executed by the processor, cause the electronic device to obtain correction data comprising an invasively measured blood glucose value and a measurement time point, learn a difference between a final blood glucose value non-invasively measured and determined by a blood glucose measuring device and the blood glucose value in the correction data to determine a corrected blood glucose value. The blood glucose measuring device is configured to obtain, via a first receiver positioned on a same side as a light source based on a body of a user, a plurality of reflection signals, which is a plurality of lights output from the light source being multiply scattered and reflected in a reverse direction from the body, and obtain, via a second receiver positioned on an opposite side of the light source, a plurality of transmission signals, which is the plurality of lights being multiply scattered and transmitted in a forward direction while passing through the body, determine, for each of the plurality of reflection signals and each of the plurality of transmission signals, a slope efficiency, which represents a ratio of an output change to an input change of the blood glucose measuring device, and determine the final blood glucose value using at least one of the slope efficiency of the plurality of reflection signals or the slope efficiency of the plurality of transmission signals. The plurality of lights has different wavelengths.

The instructions may, when executed by the processor, cause the electronic device to determine the final blood glucose value reflecting a difference between a final blood glucose value prior to obtaining the correction data and the blood glucose value in the correction data.

According to an aspect, there is provided an operating method of a blood glucose measuring device including obtaining, via a first receiver positioned on a same side as a light source based on a body of a user, a plurality of reflection signals, which is a plurality of lights output from the light source being multiply scattered and reflected in a reverse direction from the body, and obtaining, via a second receiver, a second receiver positioned on an opposite side of the light source, a plurality of transmission signals, which is the plurality of lights being multiply scattered and transmitted in a forward direction while passing through the body, determining, for each of the plurality of reflection signals and each of the plurality of transmission signals, a slope efficiency, which represents a ratio of an output change to an input change of the blood glucose measuring device, determining first analysis data based on the slope efficiency of the plurality of reflection signals and/or second analysis data based on the slope efficiency of the plurality of transmission signals, and determining a final blood glucose value based on at least one of the first analysis data or the second analysis data. The plurality of lights has different wavelengths.

The determining of the first analysis data and/or the second analysis data may include determining, for each of the plurality of reflection signals and each of the plurality of transmission signals, a normalized slope efficiency, which represents a relationship between the slope efficiency at a predetermined reference time point and the slope efficiency at a blood glucose measurement time point, and determining the first analysis data and the second analysis data based on the normalized slope efficiency.

The determining of the first analysis data and/or the second analysis data may include determining the first analysis data based on a first correlation between respective normalized slope efficiencies of the plurality of reflection signals and determining the second analysis data based on a second correlation between respective normalized slope efficiencies of the plurality of transmission signals.

The first correlation may be a difference between logarithmic values of the respective normalized slope efficiencies of the plurality of reflection signals.

The second correlation may be a difference between logarithmic values of the respective normalized slope efficiencies of the plurality of transmission signals.

The first correlation may be a sum of logarithmic values of the respective normalized slope efficiencies of the plurality of reflection signals.

The second correlation may be a sum of logarithmic values of the respective normalized slope efficiencies of the plurality of transmission signals.

The determining of the first analysis data and/or the second analysis data may include determining the first correlation and the second correlation based on a degree of blood glucose correlation according to the wavelengths of the plurality of lights.

The determining of the final blood glucose value may include determining a variance error of a first prediction value and a variance error of a second prediction value, wherein the first prediction value and the second prediction value are derived from the first analysis data and the second analysis data, respectively, and determining one of the first prediction value and the second prediction value of which the variance error is less than the other as the final blood glucose value.

The final blood glucose value may be corrected by an electronic device that learns, using correction data comprising a blood glucose value measured invasively and a measurement time point, a difference between the final blood glucose value and the blood glucose value in the correction data to determine a corrected blood glucose value.

Additional aspects of embodiments will be set forth in part in the description which follows and, in part, will be apparent from the description, or may be learned by practice of the disclosure.

According to embodiments, an electronic device may determine a blood glucose value more accurately by determining a corrected blood glucose value based on a difference between a blood glucose value of correction data in the electronic device and a final blood glucose value determined by a blood glucose measuring device.

According to an embodiment, a blood glucose measuring device may measure blood glucose of a user more effectively by using a non-invasive method, by determining a final blood glucose value based on a normalized slope efficiency in the blood glucose measuring device.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating a blood glucose measuring device that uses a non-invasive method, according to an embodiment;

FIG. 2 is a diagram illustrating light that is used to measure blood glucose, according to an embodiment;

FIG. 3 is a diagram illustrating an example of a blood glucose measuring device according to an embodiment;

FIG. 4 is a diagram illustrating a method of processing a signal so as not to be affected by noise in the signal obtained by a blood glucose measuring device, according to an embodiment;

FIG. 5 is a diagram illustrating a method of determining analysis data using a plurality of signals, according to an embodiment;

FIG. 6 is a diagram illustrating an electronic device that determines a corrected blood glucose value using correction data;

FIG. 7 is a diagram illustrating an operating method of a blood glucose measuring device, according to an embodiment; and

FIG. 8 is a diagram illustrating a blood glucose measuring device according to an embodiment.

### DETAILED DESCRIPTION

The following detailed structural or functional description is provided as an example only and various alterations and modifications may be made to the embodiments. Thus, an actual form of implementation is not construed as limited to the embodiments described herein and should be understood to include all changes, equivalents, and replacements within the idea and the technical scope of the disclosure.

As used herein, each of phrases such as “A or B,” “at least one of A and B,” “at least one of A or B,” “A, B, or C,” “at least one of A, B, and C,” “at least one of A, B, or C,” and “one or a combination of at least two of A, B, and C” may include any one of the items listed together in the corresponding one of the phrases or all possible combinations thereof. Although terms, such as first, second, and the like are used to describe various components, the components are not limited to the terms. These terms should be used only to distinguish one component from another component. For example, a first component may be referred to as a second component, and similarly, the second component may also be referred to as the first component.

It should be noted that when one component is described as being “connected,” “coupled,” or “joined” to another component, the first component may be directly connected, coupled, or joined to the second component, or a third component may be between the first and second components.

The singular forms “a,” “an,” and “the” used herein are intended to include the plural forms as well, unless the context clearly indicates otherwise. It will be further understood that the terms “comprises/comprising” and/or “includes/including,” when used herein, specify the presence of stated features, integers, steps, operations, elements, and/or components, but do not preclude the presence or addition of one or more other features, integers, steps, operations, elements, components and/or groups thereof.

Unless otherwise defined, all terms used herein including technical and scientific terms have the same meanings as those commonly understood by one of ordinary skill in the art to which this disclosure pertains. Terms such as those defined in commonly used dictionaries are to be interpreted as having a meaning that is consistent with their meaning in the context of the relevant art and are not to be interpreted in an idealized or overly formal sense unless expressly so defined herein.

Hereinafter, the embodiments are described in detail with reference to the accompanying drawings. When describing the embodiments with reference to the accompanying drawings, like reference numerals refer to like components and a repeated description related thereto is omitted.

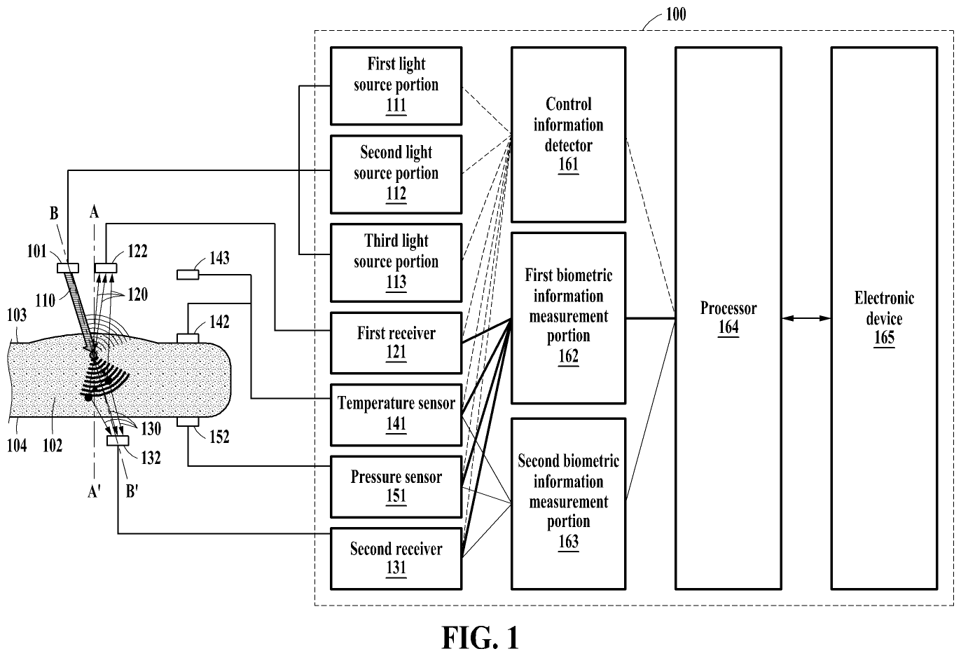

FIG. 1 is a diagram illustrating a blood glucose measuring device that uses a non-invasive method, according to an embodiment.

Referring to FIG. 1, a blood glucose measuring device 100 may include a first light source portion 111, a second light source portion 112, a third light source portion 113, a first receiver 121, a second receiver 131, a temperature sensor 141, a pressure sensor 151, a control information detector 161, a first biometric information measurement portion 162, a second biometric information measurement portion 163, and a processor 164.

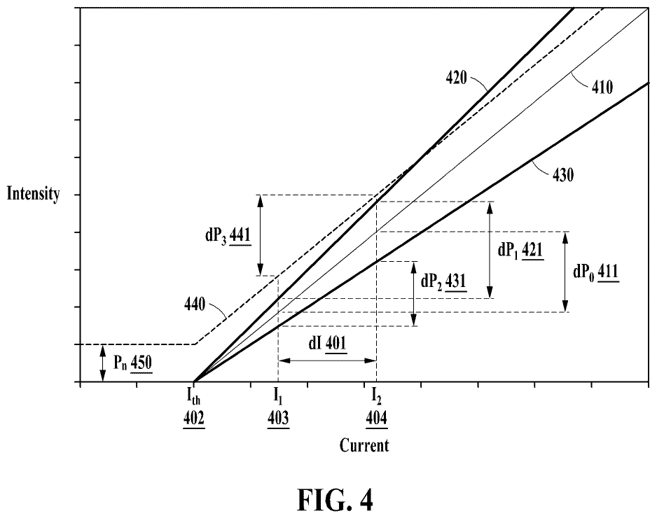

The blood glucose measuring device 100 may use an optical signal to measure blood glucose of a user using a non-invasive method. When using an optical signal, the blood glucose measuring device 100 may measure blood glucose by selectively using a wide range of wavelengths from visible light to far infrared light. For example, a wavelength in a near-infrared region may penetrate deeper into a biological tissue 105 than wavelengths in other regions and may thus exhibit optical properties according to an excellent penetration depth into the biological tissue 105, and the blood glucose measuring device 100 may effectively obtain and analyze an optical signal utilizing the optical properties. When the blood glucose measuring device 100 measures blood glucose using an optical signal, noise may occur due to external light other than a plurality of lights 110 output from a light source 101. A method of removing noise is described in more detail with reference to FIG. 4.

The blood glucose measuring device 100 may include the light source 101, the first receiver 121, and the second receiver 131. The light source 101 may output the plurality of lights 110. The plurality of lights 110 may have different wavelengths. For example, the light source 101 may output the plurality of lights 110 through the first light source portion 111, the second light source portion 112, and the third light source portion 113. Each of the plurality of lights 110 output from the first light source portion 111, the second light source portion 112, and the third light source portion 113 may have different wavelengths. Depending on the embodiment, the number of light source portions may not be limited to 3, and a greater number of light source portions may be used depending on the embodiment.

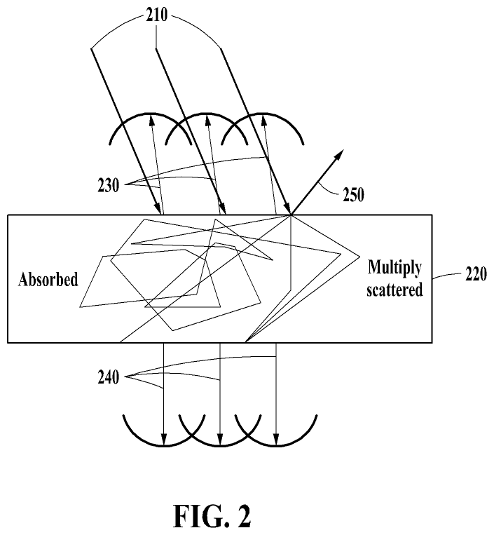

The blood glucose measuring device 100 may obtain, via the first receiver 121 positioned on a same side 103 as the light source 101 based on a body 102 of a user, a plurality of reflection signals 120, which may be a plurality of lights 110 output from the light source 101 being multiply scattered and reflected in a reverse direction from the body 102. The blood glucose measuring device 100 may obtain, via the second receiver 131 positioned on an opposite side 104 of the light source 101, a plurality of transmission signals 130, which are the plurality of lights 110 being multiply scattered and transmitted in a forward direction while passing through the body 102. The plurality of lights 110 output from the light source 101 may be multiply scattered in multiple directions without being absorbed by cells or molecules in the body 102 of the user. The plurality of reflection signals 120 may be signals among the plurality of lights 110 that are multiply scattered and reflected in the reverse direction, which is opposite to an incident direction. The plurality of transmission signals 130 may be signals among the plurality of lights 110 that are multiply scattered and transmitted in the forward direction, which is a same direction as the incident direction. Light used to measure blood glucose may be described in more detail with reference to FIG. 2. According to an embodiment, the first receiver 121 may obtain, via a first receiving node 122, a plurality of signals multiply scattered and reflected from the body 102 and may convert and amplify the obtained plurality of signals to obtain the plurality of reflection signals 120. According to an embodiment, the second receiver 131 may collect, via a second receiving node 132, a plurality of signals multiply scattered and transmitted through the body 102 and may convert and amplify the plurality of signals to obtain the plurality of transmission signals 130. For example, the first receiver 121 and the second receiver 131 may convert and amplify a plurality of current signals collected via the first receiving node 122 and the second receiving node 132, respectively, into a plurality of voltage signals to obtain the plurality of reflection signals 120 and the plurality of transmission signals 130. The first receiver 121 and the second receiver 131 may also control and detect temperature.

The blood glucose measuring device 100 may further include the temperature sensor 141 and the pressure sensor 151. The temperature sensor 141 may obtain information on a temperature change of the biological tissue 105 and a temperature change of the surrounding environment via a first temperature sensor node 142 that is in close contact with the biological tissue 105 and a separate second temperature sensor node 143, respectively. The pressure sensor 151 may obtain information on a change in pressure applied to the same side 103 as the light source 101 and the opposite side 104 from the light source 101 with respect to the body 102 of the user via one or more pressure sensor nodes 152 that are in close contact with the biological tissue 105.

The control information detector 161 may determine whether the blood glucose measuring device 100 normally obtains signals or information necessary for blood glucose measurement from the biological tissue 105, based on signals or information obtained via the first light source portion 111, the second light source portion 112, the third light source portion 113, the first receiver 121, the second receiver 131, the temperature sensor 141, and the pressure sensor 151. For example, the control information detector 161 may determine whether the blood sugar measuring device 100 is normally worn on the biological tissue 105 based on pressure information obtained via the pressure sensor 151 or may determine whether the temperature of the blood glucose measuring device 100 is within a temperature range suitable for obtaining biometric information based on temperature information obtained via the temperature sensor 141. According to an embodiment, the control information detector 161 may monitor whether a temperature, output, or intensity of a current applied to the first light source portion 111, the second light source portion 112, and the third light source portion 113 is included within a preset normal range.

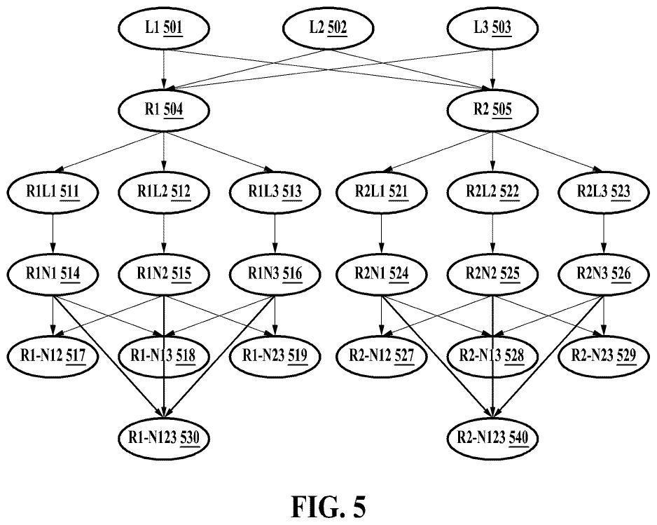

The first biometric information measurement portion 162 may generate first analysis data based on the plurality of reflection signals 120 obtained via the first receiver 121. The second biometric information measurement portion 163 may determine second analysis data based on the plurality of transmission signals 130 obtained via the second receiver 131. According to an embodiment, the first biometric information measurement portion 162 and the second biometric information measurement portion 163 may more accurately determine the first analysis data and the second analysis data based on the plurality of reflection signals 120 and the plurality of transmission signals 130 using the temperature sensor 141 and the pressure sensor 151, respectively. The processes of determining the first analysis data and the second analysis data based on the plurality of reflection signals 120 and the plurality of transmission signals 130 are described in more detail with reference to FIGS. 4 and 5, respectively.

The processor 164 may determine a final blood glucose value based on at least one of information including whether the blood glucose measuring device 100 is operating normally, the first analysis data, or the second analysis data. The information may be obtained via the control information detector 161, the first biometric information measurement portion 162, and the second biometric information measurement portion 163, respectively.

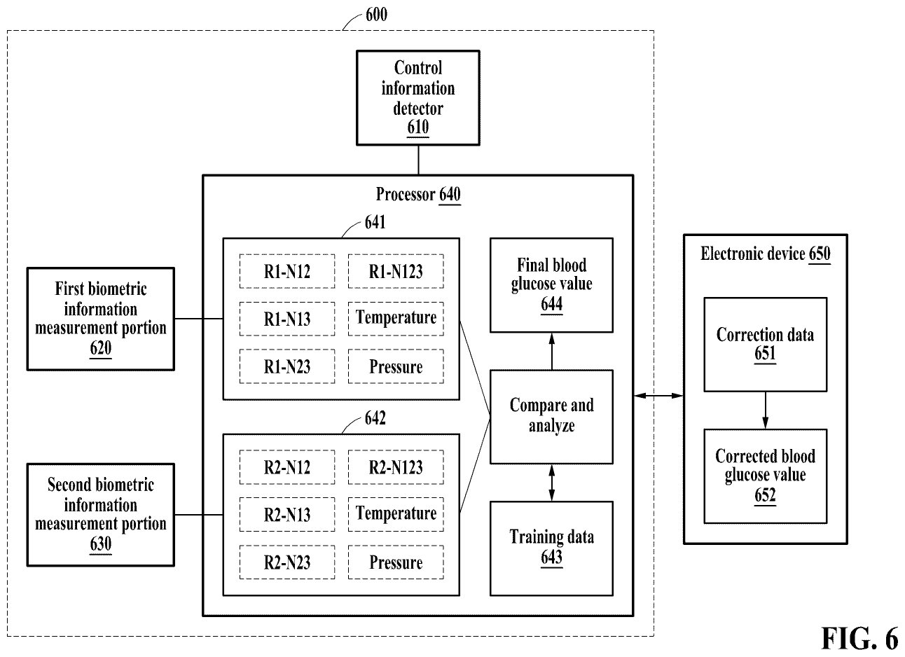

According to an embodiment, an electronic device 165 may determine a corrected blood glucose value based on the final blood glucose value determined by the blood glucose measuring device 100 and correction data input to the electronic device 165. The operation of determining the corrected blood glucose value using the electronic device 165 is described in more detail with reference to FIG. 6.

FIG. 2 is a diagram illustrating light that is used to measure blood glucose, according to an embodiment.

Referring to FIG. 2, simple reflection lights 250, a plurality of reflection signals 230, and a plurality of transmission signals 240, which may be generated by a plurality of lights 210 output from a light source being incident on a biological tissue 220, are illustrated.

Some of the plurality of lights 210 multiply scattered within the biological tissue 220 may become the plurality of reflection signals 230, which are reflected in an opposite direction to an incident direction of the plurality of lights 210. In FIG. 2, the plurality of reflection signals 230 is illustrated in a vertical direction, but the direction of the plurality of reflection signals 230 is not limited thereto, and the plurality of reflection signals 230 may be signals that are multiply scattered and reflected in the opposite direction to the incident direction of the plurality of lights 210, which is toward the biological tissue 220.

Some of the plurality of lights 210 multiply scattered within the biological tissue 220 may become the plurality of transmission signals 240, which are transmitted in a same direction as the incident direction of the plurality of lights 210. In FIG. 2, the plurality of transmission signals 240 is also illustrated in a vertical direction, but the direction of the plurality of transmission signals 240 is not limited thereto, and the plurality of transmission signals 240 may be signals that are multiply scattered and transmitted in a forward direction while passing through the biological tissue 220.

The simple reflection lights 250 may be signals reflected from a surface of the biological tissue 220 among the plurality of lights 210 output from the light source. The simple reflection lights 250 may be signals that are not absorbed or scattered within the biological tissue 220 but simply reflected from the surface of the biological tissue 220. The simple reflection lights 250 may not reflect information related to biosignals such as blood glucose and may thus not be used for blood glucose measurement.

The signals used to determine the final blood sugar value in a blood glucose measuring device may be the plurality of reflection signals 230 multiply scattered and reflected and/or the plurality of transmission signals 240 multiply scattered and transmitted, and the simple reflection lights 250 simply reflected from the surface of the biological tissue 220 may not be used. Although not illustrated in FIG. 2, among the plurality of lights 210, lights absorbed within the biological tissue 220 may not be measured by a receiver of the blood glucose measuring device.

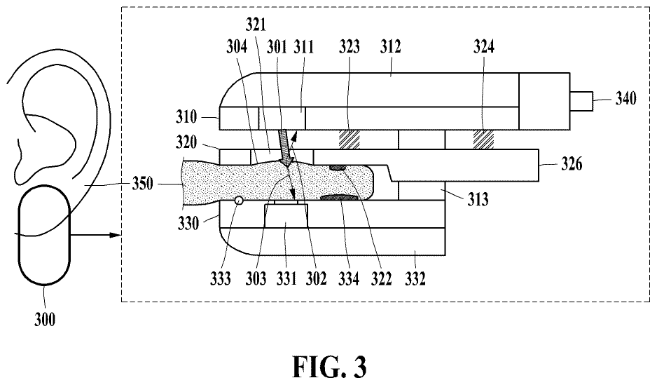

FIG. 3 is a diagram illustrating an example of a blood glucose measuring device according to an embodiment.

Referring to FIG. 3, a blood glucose measuring device 300 including a first module 310, a second module 320, and a third module 330 is illustrated. The blood glucose measuring device 300 may be worn on an earlobe 350 to measure blood glucose in a non-invasive manner.

The first module 310 included in the blood glucose measuring device 300 may include a light source 311 including the first light source portion 111, the second light source portion 112, and the third light source portion 113 described with reference to FIG. 1. The first module 310 may include a first receiver that receives a plurality of reflection signals 302, which may be a plurality of lights 301 output from the light source 311 being multiply scattered and reflected from a body. Depending on the embodiment, the first module 310 may further include a first receiving node connected to the first receiver. As the description given above with reference to FIG. 1 may apply to the first receiver and the first receiving node connected to the first receiver, a more detailed description is omitted herein. The light source 311 may irradiate the plurality of lights 301 having different wavelengths onto an upper portion of a biological tissue of the earlobe 350. According to an embodiment, the light source 311 may include the first receiver, and the plurality of reflection signals 302 multiply scattered and reflected from the upper portion within the biological tissue may be received via the first receiver.

The second module 320 may include a circular passage 321 through which the plurality of lights 301 may be input and output. The second module 320 may be supported by a pillar 313 connecting the first module 310 to the third module 330. According to an embodiment, the second module 320 may be configured to be movable in an up-down direction. The second module 320 may receive an elastic force downward by two or more elastic springs 323 and 324 positioned between the first module 310 and the second module 320. When a user inserts the earlobe 350 into the blood glucose measuring device 300, the user may press a pressing plate 326 formed on one side of the second module 320 so that the second module 320 may be brought into maximum contact with the first module 310. When the earlobe 350 is inserted between the second module 320 and the third module 330 and the pressing plate 326 is released, the earlobe 350 may be fixed as illustrated in FIG. 3 by the elastic force of the elastic springs 323 and 324. When the circular passage 321 of the second module 320 is in close contact with the earlobe 350, as illustrated in FIG. 3, a portion 304 of the earlobe 350 may protrude convexly in a direction toward the circular passage 321, and the portion 304 of the earlobe 350 may allow the blood glucose measuring device 300 to be fixed to the earlobe 350. According to an embodiment, a protrusion 322 included in the second module 320 may be in close contact with the earlobe 350 to prevent slipping.

The third module 330 may include a second receiver 331, a temperature sensor 333, and a pressure sensor 334, as described with reference to FIG. 1. Depending on the embodiment, the third module 330 may further include a second receiving node connected to the second receiver 331. As the description given above with reference to FIG. 1 may apply to the second receiver 331, the second receiving node, the temperature sensor 333, and the pressure sensor 334, a more detailed description is omitted herein.

The control information detector, the first biometric information measurement portion, the second biometric information measurement portion, and the processor described with reference to FIG. 1 may be integrated into a first portion 312 of the first module 310 and a second portion 332 of the second module 320. According to an embodiment, a cable 340 connected to the first module 310 may be connected to an external battery.

According to an embodiment, only part of the first light source portion, the second light source portion, the third light source portion, the first receiver, the second receiver 331, the temperature sensor, the pressure sensor, the control information detector, the first biometric information measurement portion, the second biometric information measurement portion, and the processor described above with reference to FIG. 1 may be included in the first portion 312 and the second portion 332.

FIG. 4 is a diagram illustrating a method of processing a signal so as not to be affected by noise in a signal obtained by a blood glucose measuring device, according to an embodiment.

Referring to FIG. 4, signals including noise 450 and signals not including the noise 450 are illustrated.

The blood glucose measuring device may determine, for each of a plurality of reflection signals and a plurality of transmission signals, a slope efficiency representing a ratio of an output change to an input change of the blood glucose measuring device. The plurality of reflection signals may be signals, which are a plurality of lights being multiply scattered and reflected in a reverse direction, and the plurality of transmission signals may be signals, which are a plurality of lights being multiply scattered and transmitted in a forward direction. In the blood glucose measuring device, an output may increase in proportion to an input applied for an input greater than or equal to a threshold input 402. The graph illustrated in FIG. 4 may represent an output change according to an input change measured by a first receiver or a second receiver for a plurality of lights incident on a biological tissue such as an earlobe. In other words, the slope efficiency may be determined as the ratio of the output change to a difference in an input applied to the blood glucose measuring device, which may correspond to a slope of a graph corresponding to the input greater than or equal to the threshold input 402. The input change may be a change in a current intensity applied to the light source, and the output change may be a change in a plurality of reflection signals and a plurality of transmission signals corresponding to the current intensity. For example, for an input change amount 401 corresponding to a difference between a first current value 403 and a second current value 404, a first signal 410, a second signal 420, and a third signal 430 may have a first output change amount 411, a second output change amount 421, and a third output change amount 431, respectively. That is, the first output change amount 411, the second output change amount 421, and the third output change amount 431 may be determined by a difference between a y value corresponding to the first current value 403 and a y value corresponding to the second current value 404. Accordingly, slope efficiencies of the first signal 410, the second signal 420, and the third signal 430 may be determined as the first output change amount 411/the input change amount 401, the second output change amount 421/the input change amount 401, and the third output change amount 431/the input change amount 401, respectively. The first signal 410, the second signal 420, and the third signal 430 may have an output intensity of 0 for inputs prior to the threshold input 402. In other words, the first signal 410, the second signal 420, and the third signal 430 may be a signal without the noise 450. The first signal 410, the second signal 420, and the third signal 430 may be signals that increase linearly according to inputs greater than or equal to the threshold input 402.

According to an embodiment, the first signal 410, the second signal 420, and the third signal 430, which may be measured at an arbitrary time point, may have an extinction coefficient determined differently depending on a blood glucose change in the biological tissue such as the earlobe. When the extinction coefficient of the second signal 420 measured at a particular blood glucose level is less than that of the first signal 410 measured at a different blood glucose level, the slope efficiency of the second signal 420 may be greater than the slope efficiency of the first signal 410, as illustrated in FIG. 4. On the contrary, when the extinction coefficient of the third signal 430 is greater than that of the first signal 410, the slope efficiency of the third signal 430 may be less than that of the first signal 410.

As described above, the method of using the slope efficiency, which represents the ratio of an output change to an input change of the blood glucose measuring device, may easily remove the noise 450 caused by an external light. For example, when the noise 450 due to the external light exists, a fourth signal 440 measured by the first receiver or the second receiver may have the noise 450 of a certain size added and may thus have a greater output than the first signal 410, the second signal 420, and the third signal 430 without the noise 450. Even when the noise 450 exists, for the input change amount 401 corresponding to the difference between the first current value 403 and the second current value 404, a slope efficiency of the fourth signal 440 may be determined as a fourth output change amount 441/the input change amount 401. Since the fourth signal 440 has the same slope as the first signal 410 and differs only in presence or absence of the noise 450, the slope efficiency of the fourth signal 440 may be determined to be the same as the slope efficiency of the first signal 410. The fourth signal 440 may also be a signal that increases linearly according to inputs greater than or equal to the threshold input 402.

The blood glucose measuring device may more effectively remove the noise 450 applied to the blood glucose measuring device by determining the slope efficiency, which represents the ratio of an output change to an input change, regardless of the presence or absence of the noise 450 caused by the external light.

FIG. 5 is a diagram illustrating a method of determining analysis data using a plurality of signals, according to an embodiment.

L1 501, L2 502, and L3 503 may be a plurality of lights that is output from a light source and has different wavelengths. Referring to FIG. 5, three wavelengths are illustrated, but the number of lights is not limited to 3, and there may be different numbers of wavelengths depending on the light source. The L1 501, the L2 502, and the L3 503 having different wavelengths may, when multiply scattered and reflected, be received via a first receiver R1 504, and when multiply scattered and transmitted, be received via a second receiver R2 505. Slope efficiencies of a plurality of reflection lights of the L1 501, the L2 502, and the L3 503 received via the first receiver R1 504 may be determined as R1L1 511, R1L2 512, and R1L3 513, respectively, according to the method described with reference to FIG. 4. Slope efficiencies of a plurality of transmission lights of the L1 501, the L2 502, and the L3 503 received via the second receiver R2 505 may be determined as R2L1 521, R2L2 522, and R2L3 523, respectively, according to the method described with reference to FIG. 4.

The blood glucose measuring device may determine first analysis data based on a slope efficiency of a plurality of reflection signals and/or second analysis data based on a slope efficiency of a plurality of transmission signals. According to an embodiment, the blood glucose measuring device may determine, for each of the plurality of reflection signals and the plurality of transmission signals, a normalized slope efficiency representing a relationship between a slope efficiency at a predetermined reference time point and a slope efficiency at a blood glucose measurement time point. For example, when the L1 501 is reflected and received via the R1 504 at a predetermined reference time point t0 and the slope efficiency is determined as R1L1(t0), a normalized slope efficiency R1N1 514 determined at an arbitrary time point t may be determined as R1L1(t)/the R1L1(t0). Similarly, when the L2 502 and the L3 503 are reflected and received via the R1 504 at the predetermined reference time point t0 and slope efficiencies thereof are determined as R1L2(t0) and R1L3(t0), respectively, normalized slope efficiencies R1N2 515 and R1N3 516 determined at the arbitrary time point t may be determined as R1L2(t)/R1L2(t0) and R1L3(t)/R1L3(t0), respectively. Depending on the embodiment, the reference time point t0 may be a time point after a predetermined period of time subsequent to a time point at which a user wears the blood glucose measuring device. When the L1 501, the L2 502, and the L3 503 are transmitted and received via the R2 505, the blood glucose measuring device may determine the slope efficiencies thereof as the R2L1 521, the R2L2 522, and the R2L3 523, respectively, and may determine the normalized slope efficiencies thereof as R2N1 524, R2L2 525, and R2N3 526, respectively. The R2N1 524, the R2N2 525, and the R2N3 526, which are the normalized slope efficiencies, may be determined as R2L1(t)/R2L1(t0), R2L2(t)/the R2L2(t0), and R2L3(t)/the R2L3(t0), respectively. By normalizing a signal at a time point to measure blood glucose with respect to a predetermined reference time, the blood sugar measuring device may determine a final blood glucose value based on signals obtained more accurately and consistently. The method of determining the normalized slope efficiency is not limited to the examples described above. The normalized slope efficiency may be determined by comparing a value at a measurement time point with a value at any fixed time point.

The blood glucose measuring device may determine first analysis data based on a first correlation between the respective normalized slope efficiencies of the plurality of reflection signals and determine second analysis data based on a second correlation between the respective normalized slope efficiencies of the plurality of transmission signals. According to an embodiment, the first correlation may be a difference between logarithmic values of the respective normalized slope efficiencies of the plurality of reflection signals, and the second correlation may be a difference between logarithmic values of the respective normalized slope efficiencies of the plurality of transmission signals. For example, R1-N12 517 may be determined as a difference between a logarithmic value of the normalized slope efficiency R1N1 514 and a logarithmic value of the normalized slope efficiency R1N2 515. Similarly, R1-N13 518, R1-N23 519, R2-N12 527, R2-N13 528, and R2-N23 529 may be determined as a difference between the logarithmic value of the R1N1 514 and a logarithmic value of the R1N3 516, a difference between the logarithmic value of the R1N2 515 and the logarithmic value of the R1N3 516, a difference between a logarithmic value of the R2N1 524 and a logarithmic value of the R2N2 525, a difference between the logarithmic value of the R2N1 524 and a logarithmic value of the R2N3 526, and a difference between the logarithmic value of the R2N2 525 and the logarithmic value of the R2N3 526, respectively.

According to an embodiment, the first correlation may be a sum of the logarithmic values of the respective normalized slope efficiencies of the plurality of reflection signals, and the second correlation may be a sum of the logarithmic values of the respective normalized slope efficiencies of the plurality of transmission signals. For example, R1-N123 530 may be a sum of the logarithmic values of the R1N1 514, the R1N2 515, and the R1N3 516, and R2-N123 540 may be a sum of the logarithmic values of the R2N1 524, the R2N2 525, and the R2N3 526.

By determining the first correlation between the respective normalized slope efficiencies of the plurality of reflection signals and the second correlation between the respective normalized slope efficiencies of the plurality of transmission signals based on the logarithmic values, the blood glucose measuring device may reduce skewness and kurtosis between pieces of data of the obtained signals, thereby improving a regularity of the final blood glucose value, and may mitigate an influence of a momentary movement of the user or external noise during continuous blood glucose measurement, thereby increasing a reliability of the final blood glucose value.

The blood glucose measuring device may determine the first correlation and the second correlation based on a degree of blood glucose correlation according to the wavelengths of the plurality of lights. The degree of blood glucose correlation may represent a degree of variability of each of a plurality of reflection signals and a plurality of transmission signals obtained according to the wavelengths. Depending on the embodiment, when at least one of a plurality of lights has a low blood glucose correlation, the blood glucose measuring device may determine the first correlation as a difference between logarithmic values of normalized slope efficiencies of a plurality of reflection signals based on the plurality of lights, and may determine the second correlation as a difference between logarithmic values of normalized slope efficiencies of a plurality of transmission signals based on the plurality of lights. Depending on the embodiment, when all of a plurality of lights have a high blood glucose correlation, the blood glucose measuring device may determine the first correlation as a sum of logarithmic values of normalized slope efficiencies of a plurality of reflection signals based on the plurality of lights, and may determine the second correlation as a difference between logarithmic values of normalized slope efficiencies of a plurality of transmission signals based on the plurality of lights. For example, when the blood glucose correlation between the L1 501, L2 502, and L3 503, which may be the plurality of lights having different wavelengths, is absent or very low, the blood glucose measuring device may determine the first correlation and the second correlation using a difference between logarithmic values of the normalized slope efficiencies of the plurality of reflection signals based on the L1 501, the L2 502, and the L3 503. When the first correlation and the second correlation are determined as the difference of the logarithmic values of the normalized slope efficiencies, common interference effects due to temperature, pressure, and movement of the blood glucose measuring device may be offset while reducing the influence due to the blood glucose correlation.

Depending on the embodiment, when a plurality of lights has a high blood glucose correlation, the blood glucose measuring device may determine the first correlation as a sum of logarithmic values of normalized slope efficiencies of a plurality of reflection signals based on the plurality of lights, and may determine the second correlation as a sum of logarithmic values of normalized slope efficiencies of a plurality of transmission signals based on the plurality of lights. For example, when the blood glucose correlation between the L1 501, L2 502, and L3 503, which may be the plurality of lights having different wavelengths, is high, the blood glucose measuring device may determine the second correlation using a sum of logarithmic values of the normalized slope efficiencies of the plurality of transmission signals based on the L1 501, the L2 502, and the L3 503. When the first correlation and the second correlation are determined as the sum of the logarithmic values of the normalized slope efficiencies, blood glucose correlations of the plurality of lights according to each wavelength may be mutually combined. When the blood glucose correlations of the plurality of lights are mutually combined, blood glucose may be effectively measured without a multicollinearity issue of the plurality of lights with a high blood glucose correlation.

The blood glucose measuring device may determine the first analysis data based on the difference between or sum of the logarithmic values of the respective normalized slope efficiencies of the plurality of reflection signals, and may determine the second analysis data based on the difference between or sum of the logarithmic values of the respective normalized slope efficiencies of the plurality of transmission signals.

The blood glucose measuring device may determine respective variance errors of a first prediction value and a second prediction value that are derived from the first analysis data and the second analysis data, respectively, and may determine one of the first prediction value and the second prediction value having a smaller variance error as the final blood glucose value. By determining, as the final blood glucose value, one of the first prediction value and the second prediction value determined via the first receiver and the second receiver, which has a smaller variance error, the blood glucose measuring device may measure the blood glucose of a user using the blood glucose measuring device more accurately in a non-invasive manner.

FIG. 6 is a diagram illustrating an electronic device that determines a corrected blood glucose value using correction data.

A blood glucose measuring device 600 may collect, via a first biometric information measurement portion 620, information on a plurality of reflection signals obtained via a first receiver and may collect, via a second biometric information measurement portion 630, information on a plurality of transmission signals obtained via a second receiver. As the description given with reference to FIG. 1 may apply to the first receiver, the second receiver, a control information detector 610, the first biometric information measurement portion 620, and the second biometric information measurement portion 630, a more detailed description is omitted herein.

The blood glucose measuring device 600 may determine, via a processor 640, first analysis data 641 and second analysis data 642 based on information received from the first biometric information measurement portion 620 and the second biometric information measurement portion 630, respectively. The first analysis data 641 may be determined based on information on R1-N12, R1-N13, R1-N23, R1-N123, a temperature of the first receiver, and a pressure of the first receiver, which may be determined based on the first correlation between the normalized slope efficiencies, and the second analysis data 642 may be determined based on information on R2-N12, R2-N13, R2-N23, R2-N123, a temperature of the second receiver, and a pressure of the second receiver, which may be determined based on the second correlation between the normalized slope efficiencies.

The blood glucose measuring device 600 may determine a final blood glucose value 644 by comparing and analyzing the first analysis data 641 and the second analysis data 642 via the processor 640. The blood glucose measuring device 600 may determine respective variance errors of a first prediction value and a second prediction value that are derived from the first analysis data 641 and the second analysis data 642, respectively, and may determine one of the first prediction value and the second prediction value having a smaller variance error as the final blood glucose value 644. The blood glucose measuring device 600 may determine the variance errors based on training data 643 pre-trained via the processor 640.

Since the blood glucose measuring device 600 may measure the blood glucose of the user in a non-invasive manner, there may be an error between the determined final blood glucose value 644 and an actual blood glucose value of a user of the blood glucose measuring device 600. To correct the errors, an electronic device 650 may correct the blood sugar value using correction data 651.

The electronic device 650 may obtain the correction data 651 that includes an invasively measured blood glucose value and a measurement time point. For example, the electronic device 650 may receive, from the user, the correction data 651 including a blood glucose value measured using collected blood of a user and a measurement time point. The correction data 651 may be more accurate than the final blood glucose value determined after measured by a non-invasive method since the correction data 651 includes a blood glucose value based on the blood of the user and a time point at which the blood is collected. Depending on the embodiment, the processor 640 of the blood glucose measuring device 600 may be included in the electronic device 650. In other words, the blood glucose measuring device 600 may obtain signals using the control information detector 610, the first biometric information measurement portion 620, and the second biometric information measurement portion 630, and the final blood glucose value 644 determined by the processor 640 may also be determined by the electronic device 650.

The electronic device 650 may learn a difference between the final blood glucose value 644 non-invasively measured and determined by the blood glucose measuring device 600 and the blood glucose value in the correction data 651 to determine a corrected blood glucose value 652. According to an embodiment, the electronic device 650 may determine the final blood glucose value by reflecting the difference between a final blood glucose value 644 at a time point prior to obtaining the correction data 651 and the blood glucose value in the correction data 651. For example, when the final blood glucose value 644 determined non-invasively using a blood glucose measurement device 600 is 60 milligrams (mg)/deciliter (dL), and the blood glucose value in the correction data 651 is 80 mg/dL, the difference between the final blood glucose value 644 and the blood glucose value in the correction data 651 may be 20 mg/dL. After receiving the correction data 651, the electronic device 650 may reflect the difference between the two values and accordingly determine a blood glucose value obtained by adding 20 mg/dL to the final blood glucose value 644, which was determined by the blood glucose measuring device 600, as a corrected blood glucose value 652. By using the correction data 651 measured in an invasive manner on a one-time basis, the electronic device 650 may improve the accuracy of the final blood glucose value 644 measured by the blood glucose measuring device 600. For example, the electronic device 650 may receive the correction data 651 from the user once a day or at preset time intervals to determine a more accurate corrected blood glucose value 652.

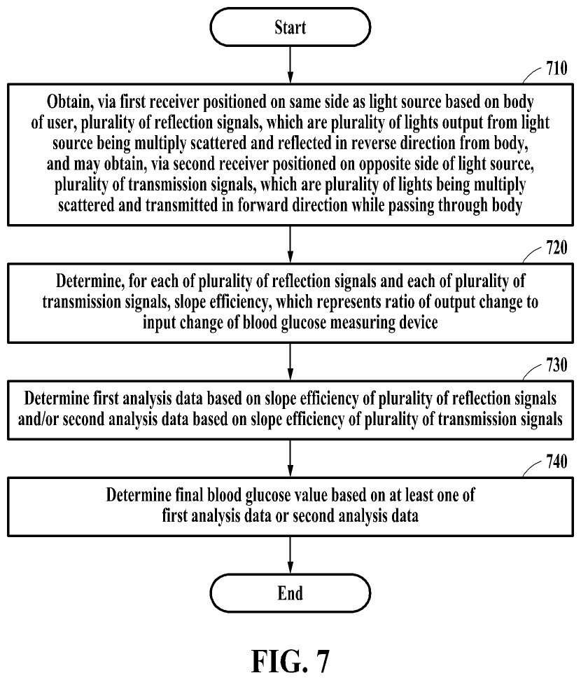

FIG. 7 is a diagram illustrating an operating method of a blood glucose measuring device, according to an embodiment.

In the following embodiments, operations may be performed sequentially, but not necessarily. For example, the order of the operations may change, and at least two of the operations may be performed in parallel. Operations 710 to 740 may be performed by at least one component (e.g., a memory or a processor) of an electronic device.

In operation 710, the blood glucose measuring device may obtain, via a first receiver positioned on a same side as a light source based on a body of a user, a plurality of reflection signals, which is a plurality of lights output from the light source being multiply scattered and reflected in a reverse direction from the body, and may obtain, via a second receiver positioned on an opposite side of the light source, a plurality of transmission signals, which is the plurality of lights being multiply scattered and transmitted in a forward direction while passing through the body. The plurality of lights may have different wavelengths.

In operation 720, the blood glucose measuring device may determine, for each of the plurality of reflection signals and each of the plurality of transmission signals, a slope efficiency, which represents a ratio of an output change to an input change of the blood glucose measuring device. The input change may be a change in a current intensity applied to the light source, and the output change may be a change in a plurality of reflection signals and a plurality of transmission signals corresponding to the current intensity.

In operation 730, the blood glucose measuring device may determine first analysis data based on the slope efficiency of the plurality of reflection signals and/or second analysis data based on the slope efficiency of the plurality of transmission signals. The operation of determining the first analysis data and/or the second analysis data may include determining, for each of the plurality of reflection signals and each of the plurality of transmission signals, a normalized slope efficiency, which represents a relationship between the slope efficiency at a predetermined reference time point and the slope efficiency at a blood glucose measurement time point, and determining the first analysis data and the second analysis data based on the normalized slope efficiency.

The operation of determining the first analysis data and/or the second analysis data may include determining the first analysis data based on a first correlation between the respective normalized slope efficiencies of the plurality of reflection signals, and determining the second analysis data based on a second correlation between the respective normalized slope efficiencies of the plurality of transmission signals. The first correlation may be a difference between logarithmic values of the respective normalized slope efficiencies of the plurality of reflection signals. The second correlation may be a difference between logarithmic values of the respective normalized slope efficiencies of the plurality of transmission signals. The first correlation may be a sum of the logarithmic values of the respective normalized slope efficiencies of the plurality of reflection signals. The second correlation may be a sum of the logarithmic values of the respective normalized slope efficiencies of the plurality of transmission signals. The operation of determining the first analysis data and/or the second analysis data may include determining the first correlation and the second correlation based on a degree of blood glucose correlation according to the wavelengths of the plurality of lights.

In operation 740, the blood glucose measuring device may determine a final blood glucose value based on at least one of the first analysis data or the second analysis data. The operation of determining the final blood glucose value may include determining respective variance errors of a first prediction value and a second prediction value that are derived from the first analysis data and the second analysis data, respectively, and determining one of the first prediction value and the second prediction value having a smaller variance error as the final blood glucose value.

As the description above with reference to FIGS. 1 to 6 may apply to each of the operations illustrated in FIG. 7, a more detailed description is omitted herein.

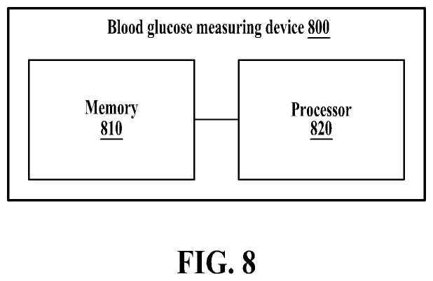

FIG. 8 is a diagram illustrating a blood glucose measuring device according to an embodiment.

Referring to FIG. 8, a blood glucose measuring device 800 may include a memory 810 and a processor 820. The memory 810 and the processor 820 may communicate with each other via a bus, peripheral component interconnect express (PCIe), and/or a network on chip (NoC).

The memory 810 may include computer-readable instructions. At least one of the instructions stored in the memory 810 may, when executed in the processor 820, cause the blood glucose measuring device 800 to perform the operations described above. The memory 810 may be volatile memory or non-volatile memory.

The processor 820 may be a device that executes instructions or programs or controls the blood glucose measuring device 800 and may include, for example, a central processing unit (CPU) and/or a graphics processing unit (GPU).

The instructions, when executed by the processor 820, may cause the blood glucose measuring device 800 to obtain, via a first receiver positioned on a same side as a light source based on a body of a user, a plurality of reflection signals, which is a plurality of lights output from the light source being multiply scattered and reflected in a reverse direction from the body, and obtain, via a second receiver positioned on an opposite side of the light source, a plurality of transmission signals, which is the plurality of lights being multiply scattered and transmitted in a forward direction while passing through the body, determine, for each of the plurality of reflection signals and each of the plurality of transmission signals, a slope efficiency, which represents a ratio of an output change to an input change of the blood glucose measuring device, determine first analysis data based on the slope efficiency of the plurality of reflection signals and/or second analysis data based on the slope efficiency of the plurality of transmission signals, and determine a final blood glucose value based on at least one of the first analysis data or the second analysis data. The plurality of lights may have different wavelengths.

The description given above may apply to other operations of the blood glucose measuring device 800.

The components described in the embodiments may be implemented by hardware components including, for example, at least one digital signal processor (DSP), a processor, a controller, an application-specific integrated circuit (ASIC), a programmable logic element, such as a field programmable gate array (FPGA), other electronic devices, or combinations thereof. At least some of the functions or the processes described in the embodiments may be implemented by software, and the software may be recorded on a recording medium. The components, the functions, and the processes described in the embodiments may be implemented by a combination of hardware and software.

The embodiments described herein may be implemented using a hardware component, a software component, and/or a combination thereof. For example, a processing device may be implemented using one or more general-purpose or special-purpose computers, such as, for example, a processor, a controller, an arithmetic logic unit (ALU), a DSP, a microcomputer, an FPGA, a programmable logic unit (PLU), a microprocessor, or any other device capable of responding to and executing instructions in a defined manner. The processing device may run an operating system (OS) and one or more software applications that run on the OS. The processing device may also access, store, manipulate, process, and create data in response to execution of the software. For purpose of simplicity, the processing device is described as singular. However, one of ordinary skill in the art will appreciate that a processing device may include multiple processing elements and/or multiple types of processing elements. For example, the processing device may include a plurality of processors, or a single processor and a single controller. In addition, a different processing configuration is possible, such as one including parallel processors.

The software may include a computer program, a piece of code, instructions, or one or more combinations thereof, to independently or collectively instruct or configure the processing device to operate as desired. Software and/or data may be embodied permanently or temporarily in any type of machine, component, physical or virtual equipment, computer storage medium or device, or in a propagated signal wave for the purpose of being interpreted by the processing device or providing instructions or data to the processing device. The software may also be distributed over network-coupled computer systems so that the software is stored and executed in a distributed fashion. The software and data may be stored in a non-transitory computer-readable recording medium.

The methods according to the embodiments may be recorded in non-transitory computer-readable media including program instructions to implement various operations of the embodiments. The media may also include the program instructions, data files, data structures, and the like alone or in combination. The program instructions recorded on the media may be those specially designed and constructed for the embodiments, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as floptical disks; and hardware devices that are specially configured to store and perform program instructions, such as read-only memory (ROM), random-access memory (RAM), flash memory, and the like. Examples of program instructions include both machine code, such as those produced by a compiler, and files containing high-level code that may be executed by the computer using an interpreter.

The above-described hardware devices may be configured to act as one or more software modules in order to perform the operations of the above-described embodiments, or vice versa.

Although the embodiments have been described with reference to the limited number of drawings, one of ordinary skill in the art may apply various technical modifications and variations based thereon. For example, suitable results may be achieved if the described techniques are performed in a different order and/or if components in a described system, architecture, device, or circuit are combined in a different manner and/or replaced or substituted by other components or their equivalents.

Therefore, other implementations, other embodiments, and equivalents to the claims are also within the scope of the following claims.

What is claimed is:

1. A blood glucose measuring device comprising:
a processor; and
a memory storing instructions,
wherein the instructions, when executed by the processor, cause the blood glucose measuring device to:
obtain, via a first receiver positioned on a same side as a light source based on a body of a user, a plurality of reflection signals, which is a plurality of lights output from the light source being multiply scattered and reflected in a reverse direction from the body, and obtain, via a second receiver positioned on an opposite side of the light source, a plurality of transmission signals, which is the plurality of lights being multiply scattered and transmitted in a forward direction while passing through the body;
determine, for each of the plurality of reflection signals and each of the plurality of transmission signals, a slope efficiency, which represents a ratio of an output change to an input change of the blood glucose measuring device;
determine first analysis data based on the slope efficiency of the plurality of reflection signals and/or second analysis data based on the slope efficiency of the plurality of transmission signals; and
determine a final blood glucose value based on at least one of the first analysis data or the second analysis data,
wherein the plurality of lights has different wavelengths.

2. The blood glucose measuring device of claim 1, wherein the instructions, when executed by the processor, cause the blood glucose measuring device to:
determine, for each of the plurality of reflection signals and each of the plurality of transmission signals, a normalized slope efficiency, which represents a relationship between the slope efficiency at a predetermined reference time point and the slope efficiency at a blood glucose measurement time point; and
determine the first analysis data and the second analysis data based on the normalized slope efficiency.

3. The blood glucose measuring device of claim 2, wherein the instructions, when executed by the processor, cause the blood glucose measuring device to:
determine the first analysis data based on a first correlation between respective normalized slope efficiencies of the plurality of reflection signals; and
determine the second analysis data based on a second correlation between respective normalized slope efficiencies of the plurality of transmission signals.

4. The blood glucose measuring device of claim 3, wherein
the first correlation is a difference between logarithmic values of the respective normalized slope efficiencies of the plurality of reflection signals, and
the second correlation is a difference between logarithmic values of the respective normalized slope efficiencies of the plurality of transmission signals.

5. The blood glucose measuring device of claim 3, wherein
the first correlation is a sum of logarithmic values of the respective normalized slope efficiencies of the plurality of reflection signals, and
the second correlation is a sum of logarithmic values of the respective normalized slope efficiencies of the plurality of transmission signals.

6. The blood glucose measuring device of claim 3, wherein the instructions, when executed by the processor, cause the blood glucose measuring device to:
determine the first correlation and the second correlation based on a degree of blood glucose correlation according to the wavelengths of the plurality of lights.

7. The blood glucose measuring device of claim 1, wherein the instructions, when executed by the processor, cause the blood glucose measuring device to:
determine a variance error of a first prediction value and a variance error of a second prediction value, wherein the first prediction value and the second prediction value are derived from the first analysis data and the second analysis data, respectively; and
determine one of the first prediction value and the second prediction value of which the variance error is less than the other as the final blood glucose value.

8. The blood glucose measuring device of claim 1, wherein
the final blood glucose value is corrected by an electronic device that learns, using correction data comprising a blood glucose value measured invasively and a measurement time point, a difference between the final blood glucose value and the blood glucose value in the correction data to determine a corrected blood glucose value.

9. The blood glucose measuring device of claim 1, wherein
the input change is a change in a current intensity applied to the light source, and
the output change is a change in a plurality of reflection signals and a plurality of transmission signals corresponding to the current intensity.

10. An electronic device comprising:
a processor; and
a memory storing instructions,
wherein the instructions, when executed by the processor, cause the electronic device to:
obtain correction data comprising an invasively measured blood glucose value and a measurement time point; and
learn a difference between a final blood glucose value non-invasively measured and determined by a blood glucose measuring device and the blood glucose value in the correction data to determine a corrected blood glucose value, and
wherein the blood glucose measuring device is configured to:
obtain, via a first receiver positioned on a same side as a light source based on a body of a user, a plurality of reflection signals, which is a plurality of lights output from the light source being multiply scattered and reflected in a reverse direction from the body, and obtain, via a second receiver positioned on an opposite side of the light source, a plurality of transmission signals, which is the plurality of lights being multiply scattered and transmitted in a forward direction while passing through the body;
determine, for each of the plurality of reflection signals and each of the plurality of transmission signals, a slope efficiency, which represents a ratio of an output change to an input change of the blood glucose measuring device; and
determine the final blood glucose value using at least one of the slope efficiency of the plurality of reflection signals or the slope efficiency of the plurality of transmission signals.

11. The electronic device of claim 10, wherein the instructions, when executed by the processor, cause the electronic device to:
determine the final blood glucose value reflecting a difference between a final blood glucose value prior to obtaining the correction data and the blood glucose value in the correction data.

12. An operating method of a blood glucose measuring device, the operating method comprising:
obtaining, via a first receiver positioned on a same side as a light source based on a body of a user, a plurality of reflection signals, which is a plurality of lights output from the light source being multiply scattered and reflected in a reverse direction from the body, and obtaining, via a second receiver, a second receiver positioned on an opposite side of the light source, a plurality of transmission signals, which is the plurality of lights being multiply scattered and transmitted in a forward direction while passing through the body;
determining, for each of the plurality of reflection signals and each of the plurality of transmission signals, a slope efficiency, which represents a ratio of an output change to an input change of the blood glucose measuring device;
determining first analysis data based on the slope efficiency of the plurality of reflection signals and/or second analysis data based on the slope efficiency of the plurality of transmission signals; and
determining a final blood glucose value based on at least one of the first analysis data or the second analysis data,
wherein the plurality of lights has different wavelengths.

13. The operating method of claim 12, wherein the determining of the first analysis data and/or the second analysis data comprises:
determining, for each of the plurality of reflection signals and each of the plurality of transmission signals, a normalized slope efficiency, which represents a relationship between the slope efficiency at a predetermined reference time point and the slope efficiency at a blood glucose measurement time point; and
determining the first analysis data and the second analysis data based on the normalized slope efficiency.

14. The operating method of claim 13, wherein the determining of the first analysis data and/or the second analysis data comprises:
determining the first analysis data based on a first correlation between respective normalized slope efficiencies of the plurality of reflection signals; and
determining the second analysis data based on a second correlation between respective normalized slope efficiencies of the plurality of transmission signals.

15. The operating method of claim 14, wherein
the first correlation is a difference between logarithmic values of the respective normalized slope efficiencies of the plurality of reflection signals, and
the second correlation is a difference between logarithmic values of the respective normalized slope efficiencies of the plurality of transmission signals.

16. The operating method of claim 14, wherein
the first correlation is a sum of logarithmic values of the respective normalized slope efficiencies of the plurality of reflection signals, and
the second correlation is a sum of logarithmic values of the respective normalized slope efficiencies of the plurality of transmission signals.

17. The operating method of claim 14, wherein the determining of the first analysis data and/or the second analysis data comprises:
determining the first correlation and the second correlation based on a degree of blood glucose correlation according to the wavelengths of the plurality of lights.

18. The operating method of claim 12, wherein the determining of the final blood glucose value comprises:
determining a variance error of a first prediction value and a variance error of a second prediction value, wherein the first prediction value and the second prediction value are derived from the first analysis data and the second analysis data, respectively; and
determining one of the first prediction value and the second prediction value of which the variance error is less than the other as the final blood glucose value.

19. The operating method of claim 12, wherein
the final blood glucose value is corrected by an electronic device that learns, using correction data comprising a blood glucose value measured invasively and a measurement time point, a difference between the final blood glucose value and the blood glucose value in the correction data to determine a corrected blood glucose value.

20. A non-transitory computer-readable storage medium storing instructions that, when executed by a processor, cause the processor to perform the method of claim 12.
